(19) United States
(12) Patent Application Publication
(10) Pub. No.: US 2015/0289897 A1
(43) Pub. Date: Oct. 15, 2015

(54) FLUID EJECTION DEVICE

(71) Applicant: SEIKO EPSON CORPORATION, Tokyo (JP)

(72) Inventors: Masaki GOMI, Hino-shi (JP); Kazuaki UCHIDA, Fujimi-machi (JP)

(21) Appl. No.: 14/682,946

(22) Filed: Apr. 9, 2015

(30) Foreign Application Priority Data
Apr. 10, 2014 (JP) 2014-080830

(51) Int. Cl. A61B 17/3203; A61M 39/22; A61M 3/02

(52) U.S. Cl. CPC A61B 17/3203; A61M 3/02; A61M 39/22; A61B 2017/00119

(57) ABSTRACT

A fluid ejection device includes a fluid accommodation portion that has a fluid outlet connected to a fluid connection tube having a channel. A fluid pressing unit causes the fluid to flow out of the fluid outlet. A fluid ejection unit ejects in a pulsed manner fluid received from the fluid connection tube. A channel opening and closing unit opens and closes the channel of the fluid connection tube. A pressure detection unit detects pressure when the fluid pressing unit operates. A control unit causes the fluid pressing unit to operate in a state in which the channel is closed, and determines that the pressure detection unit has failed depending on whether a time required until the pressure is a second determination value after the detected pressure is equal to or greater than a first determination value is equal to or greater than a predetermined determination time.

This application claims the benefit of Japanese Patent Application No. 2014-080830, filed on Apr. 10, 2014. The content of the aforementioned application is incorporated herein by reference in its entirety.

### BACKGROUND

1. Technical Field

The present invention relates to a fluid ejection device.

2. Related Art

A technology in which an object is incised or excised by ejecting a pulsed fluid is known. For example, in the medical field, a fluid ejection device is proposed as an operation scalpel to incise or excise living tissue, the fluid ejection device being configured to include a pulsation generator that ejects a pulsed fluid, a fluid supply unit that supplies a fluid to the pulsation generator, and a fluid supply path from the fluid supply unit to the pulsation generator (refer to JP-A-2013-213422).

In such a device, it is important to maintain the fluid in the fluid supply path at a proper pressure, and allow the fluid to be ejected at a suitable intensity from the pulsation generator. For this reason, the fluid ejection device has a pressure sensor for detecting the pressure of the fluid in the fluid supply path, but there is a possibility of the failure of the pressure sensor.

As examples of the failure occurring in the pressure sensor, the pressure is not detected at all because an output signal is no longer output due to a disconnection or a short circuit, and meanwhile, although the pressure is detected, the output characteristics of the output signal are shifted.

If the output characteristics of the output sensor are shifted, the pressure of the fluid in the fluid supply path is controlled in a shifted state, and thus the fluid becomes to be ejected from the pulsation generator at an intensity different from the original intensity.

### SUMMARY

An advantage of some aspects of the invention is to provide a technology for realizing a safer and highly reliable fluid ejection device that detects the failure of such a pressure sensor.

A fluid ejection device according to an aspect of the invention includes: a fluid container that has a fluid accommodation portion for accommodating a fluid, and a fluid outlet formed in the fluid accommodation portion; a fluid pressing unit that presses the fluid accommodation portion to cause the fluid to flow from the fluid outlet; a connection tube, one end of which is connected to the fluid outlet; a fluid ejection unit that has a fluid intake port connected to the other end of the connection tube, and ejects the fluid taken from the fluid intake port in a pulsed manner; a channel opening and closing unit that opens and closes a channel of the fluid in the connection tube; a pressure detection unit that detects a pressure when the fluid pressing unit presses the fluid accommodation portion, and outputs a detection signal of a level corresponding to the pressure; and a control unit that causes the fluid pressing unit to press the fluid accommodation portion in a state of causing the channel opening and closing unit to close the channel, and determines a failure of the pressure detection unit depending on whether or not a time required until the pressure is a second determination value after the pressure indicated by the detection signal is equal to or greater than a first determination value is equal to or greater than a predetermined determination time.

Other features of the present invention will become apparent from the description of the present specification and the accompanying drawings.

### BRIEF DESCRIPTION OF THE DRAWINGS

The invention will be described with reference to the accompanying drawings, wherein like numbers reference like elements.

FIG. 1 is a block diagram illustrating an example of the entire configuration of a fluid ejection device according to an embodiment of the invention.

FIG. 2 is a block diagram illustrating another example of the entire configuration of the fluid ejection device according to the embodiment of the invention.

FIG. 3 is a block diagram illustrating the configuration of a pump according to the embodiment of the invention.

FIG. 4 is a cross-sectional view illustrating the structure of a pulsation generator according to the embodiment of the invention.

FIG. 5 is a plan view illustrating the shape of an inlet channel according to the embodiment of the invention.

FIG. 6 is a block diagram illustrating the configuration of a pump control unit according to the embodiment of the invention.

FIG. 7 is a diagram illustrating a transition of a level of a detection signal of a pressure sensor according to the embodiment of the invention.

FIG. 8 is a flowchart illustrating a flow of a process of a pump control unit according to the embodiment of the invention.

FIG. 9 is a flowchart illustrating a flow of a process of the pump control unit according to the embodiment of the invention.

FIG. 10 is a diagram illustrating a configuration example of a slider according to the embodiment of the invention.

### DESCRIPTION OF EXEMPLARY EMBODIMENTS

### Outline

At least the following facts are apparent from this specification and the accompanying drawings.

A fluid ejection device includes: a fluid container that has a fluid accommodation portion for accommodating a fluid, and a fluid outlet formed in the fluid accommodation portion; a fluid pressing unit that presses the fluid accommodation portion to cause the fluid to flow from the fluid outlet; a connection tube, one end of which is connected to the fluid outlet; a fluid ejection unit that has a fluid intake port connected to the other end of the connection tube, and ejects the fluid taken from the fluid intake port in a pulsed manner; a channel opening and closing unit that opens and closes a channel of the fluid in the connection tube; a pressure detection unit that detects a pressure when the fluid pressing unit presses the fluid accommodation portion, and outputs a detection signal of a level corresponding to the pressure; and a control unit that causes the fluid pressing unit to press the fluid accommodation portion in a state of causing the channel opening and closing unit to close the channel, and determines a failure of the pressure detection unit depending on whether or not a time required until the pressure is a second determination value after the pressure indicated by the detection signal is equal to or greater than a first determination value is equal to or greater than a predetermined determination time.

According to the fluid ejection device, it is possible to realize a safer and highly reliable fluid ejection device that detects the failure of the pressure detection unit.

In the fluid ejection device, it is preferable that the fluid pressing unit includes a moving object that moves in a pressing direction of the fluid accommodation portion, and performs pressing to the fluid accommodation portion, and a position detection unit that detects a position of the moving object. Preferably, the first determination value is a value that is determined based on the pressure indicated by the detection signal which is output by the pressure detection unit when the moving object is in a predetermined position where the moving object does not come into contact with the fluid accommodation portion.

According to the fluid ejection device, even if a pressure as a reference when performing the failure detection of the pressure detection unit is shifted from normal characteristics, it is possible to detect the failure of the pressure detection unit.

In the fluid ejection device, it is preferable that the first determination value is a sum of the pressure indicated by the detection signal which is output by the pressure detection unit when the moving object is in the predetermined position and a predetermined value which is determined in accordance with an error of the pressure.

According to the fluid ejection device, when the failure of the pressure detection unit is detected, it is possible to eliminate the influence of an error of the pressure detection unit.

It is preferable that the fluid ejection device further includes a first of the pressure detection unit and a second of the pressure detection unit, the first of the pressure detection unit and the second of the pressure detection unit are arranged so as to be stacked in the pressing direction of the moving object, in a position where the first of the pressure detection unit and the second of the pressure detection unit are clamped by the moving object and the fluid accommodation portion, when the moving object presses the fluid accommodation portion, and the control unit controls the movement of the moving object by using either or both of a first of the detection signal which is output from the first of the pressure detection unit and a second of the detection signal which is output from the second of the pressure detection unit in such a manner that a pressure when the moving object presses the fluid accommodation portion is a predetermined target pressure value, continues the control by using the second of the detection signal when a failure of the first of the pressure detection unit is determined, and continues the control by using the first of the detection signal when a failure of the second of the pressure detection unit is determined.

According to the fluid ejection device, even when a failure occurs in the first pressure detection unit or the second pressure detection unit, the fluid ejection device is able to perform control to continue the ejection of the fluid from the fluid ejection unit, which allows improving the reliability of the fluid ejection device.

In the fluid ejection device, it is preferable that the control unit causes the channel opening and closing unit to open the channel, when a failure of the pressure detection unit is determined.

According to the fluid ejection device, even if the pressure in the fluid accommodation portion has become higher than an expected pressure, it is possible to reduce the pressure in the fluid accommodation portion by allowing the fluid in the fluid accommodation portion to flow from a fluid ejection unit, which enables improving the safety of the fluid ejection device.

In the fluid ejection device, it is preferable that the control unit outputs an alarm indicating a failure of the pressure detection unit, when the failure of the pressure detection unit is determined.

According to the fluid ejection device, it is possible to quickly inform an operator that the pressure detection is in a state of failure and further improve the safety of the fluid ejection device.

### Entire Configuration

Hereinafter, an embodiment of the invention will be described with reference to the accompanying drawings. A fluid ejection device according to the embodiment can be used in various procedures such as the cleaning or cutting of a fine object or structure, living tissue, or the like; however, an example of the embodiment given in the following description is the fluid ejection device suitable for use as an operation scalpel to incise or excise living tissue. Accordingly, a fluid used in the fluid ejecting device according to the embodiment is water, physiologic saline, a predetermined fluid medicine, or the like. The drawings referenced in the following description are schematic views in which a portion or a member is vertically and horizontally scaled differently from an actual scale for illustrative purposes.

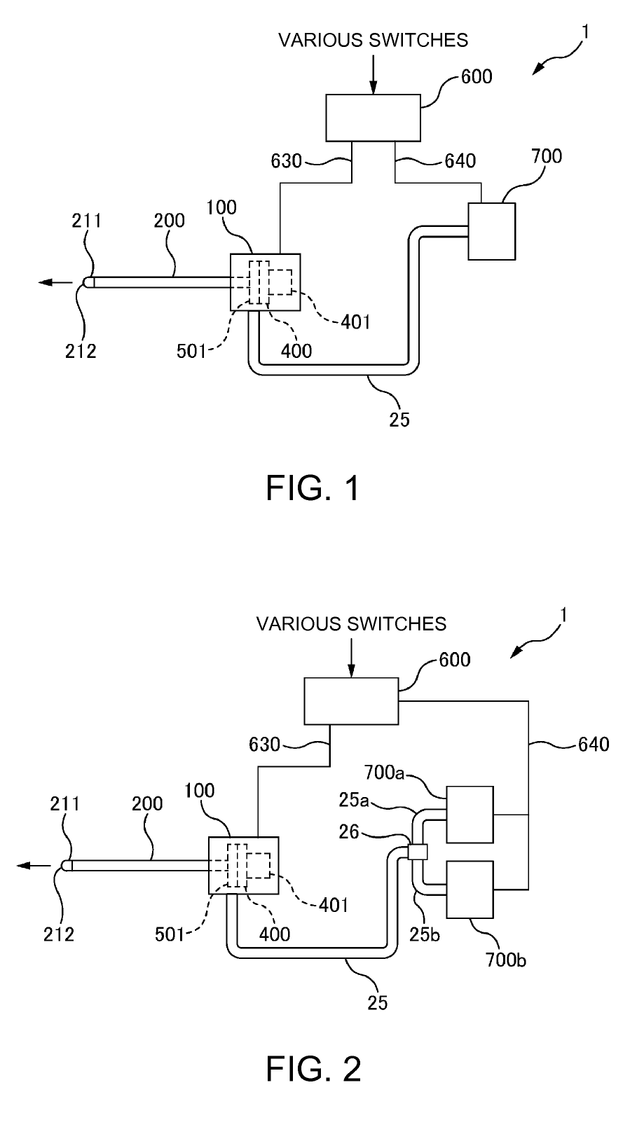

FIG. 1 is a view illustrating the configuration of a fluid ejection device 1 as an operation scalpel according to the embodiment. The fluid ejection device 1 according to the embodiment includes a pump 700 for supplying a fluid; a pulsation generator (a fluid ejection unit) 100 that converts a flow of the fluid supplied from the pump 700 into a pulsed flow, and ejects the fluid in a pulsed manner; a drive control unit 600 that controls the fluid ejection device 1 in cooperation with the pump 700; and a connection tube (connection path) 25 acting as a channel through which the pump 700 and the pulsation generator 100 are connected to each other, and the fluid flows.

The pulsation generator 100 includes a fluid chamber 501 that accommodates the fluid supplied from the pump 700; a diaphragm 400 that changes the volume of the fluid chamber 501; and a piezoelectric element 401 that vibrates the diaphragm 400, all of which will be described later in detail.

The pulsation generator 100 includes a thin pipe-like fluid ejection tube 200 that acts as a channel of the fluid discharged from the fluid chamber 501, and a nozzle 211 that is mounted on a tip end portion of the fluid ejection tube 200 and has a reduced channel diameter.

The pulsation generator 100 converts a flow of the fluid into a pulsed flow by applying a pulsed pressure to the fluid via the driving of the piezoelectric element 401 in response to drive signals output from the drive control unit 600 and the changing of the volume of the fluid chamber 501, and the pulsation generator 100 ejects the fluid in a pulsed manner via the fluid ejection tube 200 and the nozzle 211.

The drive control unit 600 and the pulsation generator 100 are connected to each other via a control cable 630, and drive signals for driving the piezoelectric element 401 are output from the drive control unit 600, and are transmitted to the pulsation generator 100 via the control cable 630.

The drive control unit 600 and the pump 700 are connected to each other via a communication cable 640, and the drive control unit 600 and the pump 700 transmit and receive various commands or data therebetween according to a predetermined communication protocol such as a controller area network (CAN).

The drive control unit 600 receives signals from various switches operated by a practitioner who performs an operation using the pulsation generator 100, and controls the pump 700 or the pulsation generator 100 via the control cable 630 or the communication cable 640.

The switches that input signals to the drive control unit 600 are a pulsation generator start-up switch 625, an ejection intensity switching switch 627, a flushing switch 628, and the like (not illustrated).

The pulsation generator start-up switch 625 is a switch (on/off) for switching between the ejection and the non-ejection of the fluid from the fluid ejection unit 100. When a practitioner who performs an operation using the pulsation generator 100 operates the pulsation generator start-up switch 625, the drive control unit 600 controls the pulsation generator 100 to eject the fluid or stop the ejection of the fluid in cooperation with the pump 700. The pulsation generator start-up switch 625 can be a switch configured to be operated by the practitioner's feet, or a switch that is provided integrally with the pulsation generator 100 grasped by the practitioner, and configured to be operated by the practitioner's hands or fingers.

The ejection intensity switching switch 627 is a switch for changing the ejection intensity of the fluid ejected from the pulse generator 100. When the ejection intensity switching switch 627 is operated, the drive control unit 600 performs a control to increase or decrease the ejection intensity of the fluid for the pulse generator 100 and the pump 700.

The flushing switch 628 will be described later.

In the embodiment, a pulsed flow implies a flow of a fluid, a flow direction of which is constant, and the flow rate or flow speed of which is changed periodically or non-periodically. The pulsed flow may be an intermittent flow in which the flowing and stopping of the fluid are repeated; however, since the flow rate or flow speed of the fluid is preferably changed periodically or non-periodically, the pulsed flow is not necessarily an intermittent flow.

Similarly, the ejection of a fluid in a pulsed form implies the ejection of the fluid by which the flow rate or moving speed of an ejected fluid is changed periodically or non-periodically. An example of the pulsed ejection is an intermittent ejection by which the ejection and non-ejection of a fluid are repeated; however, since the flow rate or moving speed of an ejected fluid is preferably changed periodically or non-periodically, the pulsed ejection is not necessarily an intermittent ejection.

When the driving of the pulsation generator 100 is stopped, that is, when the volume of the fluid chamber 501 is not changed, the fluid supplied from the pump 700 as a fluid supply unit at a predetermined pressure continuously flows out of the nozzle 211 via the fluid chamber 501.

The fluid ejection device 1 according to the embodiment may be configured to include a plurality of the pumps 700. FIG. 2 illustrates an example of the configuration of the fluid ejection device 1 configured to include two pumps 700.

In this case, as illustrated in FIG. 2, the fluid ejection device 1 includes a first pump 700a and a second pump 700b. A first connection tube 25a, a second connection tube 25b, the connection tube 25, and a three way stopcock 26 form a connection path (connection tube) which connects the pulsation generator 100 and the first pump 700a, and the pulsation generator 100 and the second pump 700b, and acts as a channel through which the fluid flows.

The three way stopcock 26 is a valve configured to be able to communicate the first connection tube 25a and the connection tube 25, or the second connection tube 25b and the connection tube 25, and either one of the first pump 700a and the second pump 700b is selectively used.

In this configuration, for example, when the first pump 700a cannot supply the fluid for unknown reasons such as a malfunction while being selected and used, it is possible to continuously use the fluid ejection device 1 and to minimize adverse effects associated with the non-supply of the fluid from the first pump 700a by switching the three way stopcock 26 so as to communicate the second connection tube 25b and the connection tube 25, and starting the supply of the fluid from the second pump 700b.

When the fluid ejection device 1 is configured to include a plurality of the pumps 700, but the pumps 700 are not required to be distinctively described, in the following description, the pumps 700 are collectively expressed by the pump 700.

In contrast, when the plurality of pumps 700 are required to be distinctively described, suffixes such as “a” and “b” are properly added to reference sign 700 of the pump, and each of the pumps 700 is distinctively expressed by the first pump 700a or the second pump 700b. In this case, each configuration element of the first pump 700a is expressed by adding the suffix “a” to a reference sign of each configuration element, and each configuration element of the second pump 700b is expressed by adding the suffix “b” to a reference sign of each configuration element.

### Pump

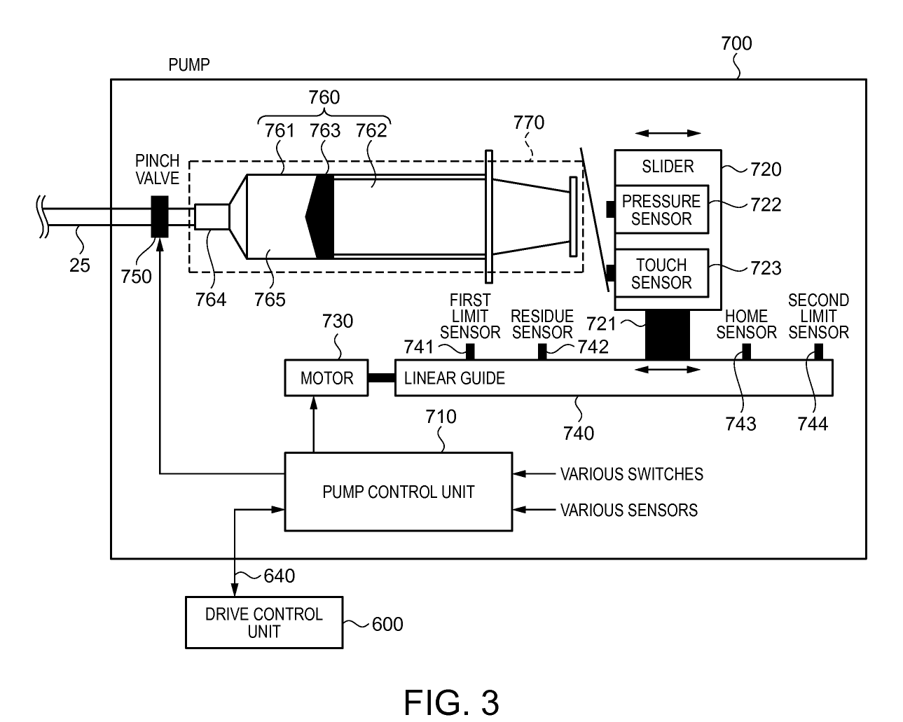

Subsequently, an outline of the configuration and operation of the pump 700 according to the embodiment will be described with reference to FIG. 3.

The pump 700 according to the embodiment includes a pump control unit (a control unit) 710; a slider (a moving object) 720; a motor 730; a linear guide 740; and a pinch valve (a channel opening and closing unit) 750. The pump 700 is configured to have a fluid container mounting unit 770 for attachably and detachably mounting a fluid container 760 that accommodates the fluid. The fluid container mounting unit 770 is formed so as to hold the fluid container 760 at a specific position when the fluid container 760 is mounted thereon.

The following switches (which will be described later in detail) (not illustrated) input signals to the pump control unit 710: a slider release switch 780; a slider set switch 781; a fluid supply ready switch 782; a priming switch 783; and a pinch valve switch 785.

In the embodiment, for example, the fluid container 760 is formed of a medical syringe configured to include a syringe 761 and a plunger 762.

In the fluid container 760, a protrusive cylinder-shaped opening (a fluid outlet) 764 is formed in a tip end portion of the syringe 761. When the fluid container 760 is mounted on the fluid container mounting unit 770, an end portion of the connection tube 25 is inserted into the opening 764, and a fluid channel is formed from the inside of the syringe 761 to the connection tube 25.

The pinch valve 750 is a valve that is provided in a path of a connection tube 25, and opens and closes a fluid channel between the fluid container 760 and the pulsation generator 100.

The pump control unit 710 controls the opening and closing of the pinch valve 750. When the pump control unit 710 opens the pinch valve 750, the fluid container 760 and the pulsation generator 100 communicate with each other via the channel therebetween. When the pump control unit 710 closes the pinch valve 750, the channel between the fluid container 760 and the pulsation generator 100 is shut off.

In a state where the fluid container 760 is mounted on the fluid container mounting unit 770, and the pinch valve 750 is opened, when the plunger 762 of the fluid container 760 moves in a direction (hereinafter, also referred to as a push-in direction or a pressing direction) in which the plunger 762 is pushed into the syringe 761, the volume of a space (hereinafter, also referred to as a fluid accommodation portion 765) is reduced, the space being enveloped by an end surface of a gasket 763 made of resin such as elastic rubber and mounted at the tip of the plunger 762 in the push-in direction, and an inner wall of the syringe 761, and the fluid in the fluid accommodation portion 765 is discharged via the opening 764 of the tip end portion of the syringe 761. The connection tube 25 is filled with the fluid discharged via the opening 764, and the discharged fluid is supplied to the pulsation generator 100.

In contrast, in a state where the fluid container 760 is mounted on the fluid container mounting unit 770, and the pinch valve 750 is closed, when the plunger 762 of the fluid container 760 moves in the push-in direction, it is possible to reduce the volume of the fluid accommodation portion 765, the fluid accommodation portion 765 being enveloped by the gasket 763 mounted at the tip of the plunger 762 and the inner wall of the syringe 761, and it is possible to increase the pressure of the fluid in the fluid accommodation portion 765.

The pump control unit 710 moves the slider 720 along a direction (in the push-in direction and the opposite direction of the push-in direction) in which the plunger 762 moves in a state where the fluid container 760 is mounted on the fluid container mounting unit 770, and the plunger 762 moves in accordance with the movement of the slider 720.

Specifically, the slider 720 is attached to the linear guide 740 in such a manner that a pedestal 721 of the slider 720 engages with a rail (not illustrated) formed linearly on the linear guide 740 along the slide direction of the plunger 762. The linear guide 740 moves the pedestal 721 of the slider 720 along the rail using power transmitted from the motor 730 driven by the pump control unit 710, and thereby the slider 720 moves along the slide direction of the plunger 762.

As illustrated in FIG. 3, the following sensors are provided along the rail of the linear guide 740: a first limit sensor 741; a residue sensor 742; a home sensor (position detector) 743; and a second limit sensor 744.

All of the first limit sensor 741, the residue sensor 742, the home sensor 743, and the second limit sensor 744 are sensors for detecting the position of the slider 720 that moves on the rail of the linear guide 740, and signals detected by these sensors are input to the pump control unit 710.

The home sensor 743 is a sensor used to determine an initial position (hereinafter, also referred to as a home position) of the slider 720 on the linear guide 740. The home position is a position in which the slider 720 is held when the fluid container 760 is mounted or replaced. The home position is set to a position where the slider 720 does not come into contact with the plunger 762, when the slider 720 is in the home position.

The residue sensor 742 is a sensor for detecting the position (hereinafter, also referred to as a residual position) of the slider 720 when the residue of the fluid in the fluid container 760 is less than or equal to a predetermined value while the slider 720 moves from the home position in the push-in direction of the plunger 762. When the slider 720 reaches the residual position in which the residue sensor 742 is provided, a predetermined alarm is output to an operator (a practitioner or an assistant). The fluid container 760 currently in use is replaced with a new fluid container 760 at an appropriate time determined by the operator. Alternatively, when an auxiliary second pump 700b having the same configuration as that of the pump 700 (the first pump 700a) is prepared, a switching operation is performed so as to supply the fluid from the auxiliary second pump 700b to the pulsation generator 100.

The first limit sensor 741 indicates a limit position (hereinafter, referred to as a first limit position) in a movable range in which the slider 720 can move from the home position in the push-in direction of the plunger 762. When the slider 720 reaches the first limit position in which the first limit sensor 741 is provided, the residue of the fluid in the fluid container 760 is much less than the residue indicating that the slider 720 is present at the residual position, and a predetermined alarm is output to the operator. In this case, the fluid container 760 currently in use is also replaced with a new fluid container 760, or a switching operation is also performed so as to supply the fluid from an auxiliary second pump 700b.

In contrast, the second limit sensor 744 indicates a limit position (hereinafter, also referred to as a second limit position) in a movable range in which the slider 720 can move from the home position in the opposite direction of the push-in direction of the plunger 762. When the slider 720 reaches the second limit position in which the second limit sensor 744 is provided, a predetermined alarm is output.

A touch sensor 723 and a pressure sensor (a pressure detection unit) 722 are mounted on the slider 720.

The touch sensor 723 is a sensor for detecting whether the slider 720 is in contact with the plunger 762 of the fluid container 760.

The pressure sensor 722 is a sensor that detects the pressure of the fluid in the fluid accommodation portion 765 formed by the inner wall of the syringe 761 and the gasket 763, that is, a pressure when the slider 720 presses the fluid accommodation portion 765, and outputs signals (detection signals) at a level (for example, a voltage, or a voltage and a frequency) that corresponds to a detected pressure.

When the pinch valve 750 is closed, and the slider 720 moves in the push-in direction, and after the slider 720 comes into contact with the plunger 762, the pressure of the fluid in the fluid accommodation portion 765 increases to the extent that the slider 720 moves further in the push-in direction.

In contrast, when the pinch valve 750 is opened, and the slider 720 moves in the push-in direction, and even after the slider 720 comes into contact with the plunger 762, the fluid in the fluid accommodation portion 765 flows out of the nozzle 211 of the pulsation generator 100 via the connection tube 25, and thereby the pressure of the fluid in the fluid accommodation portion 765 increases to a certain level, but the pressure of the fluid does not increase even though the slider 720 moves further in the push-in direction.

The touch sensor 723 and the pressure sensor 722 input signals to the pump control unit 710.

In the following description, the slider 720, the motor 730, and the linear guide 740 may be referred to as a fluid pressing unit 731. The fluid pressing unit 731 causes the fluid to flow out of the opening (the fluid outlet) 764 of the fluid container 760 by pressing the fluid accommodation portion 765.

A description to be given hereinafter is regarding a preparation operation configured to include a process of mounting a fluid container 760 filled with the fluid on the fluid container mounting unit 770; a process of supplying the fluid in the fluid container 760 to the pulsation generator 100; and a process of bringing the fluid ejection device 1 into a state in which the pulsation generator 100 can eject the fluid in the form of a pulsed flow.

First, the operator inputs an ON signal of the slider release switch 780 to the pump control unit 710 by operating the slider release switch 780. Thus, the pump control unit 710 moves the slider 720 to the home position.

The operator mounts the fluid container 760 connected to the connection tube 25 in advance on the fluid container mounting unit 770. The syringe 761 of the fluid container 760 is already filled with the fluid.

When the operator sets the connection tube 25 to the pinch valve 750, and then inputs an ON signal of the pinch valve switch 785 to the pump control unit 710 by operating the pinch valve switch 785, the pump control unit 710 closes the pinch valve 750.

Subsequently, the operator inputs an ON signal of the slider set switch 781 to the pump control unit 710 by operating the slider set switch 781. Thus, the pump control unit 710 starts a control operation in such a manner that the slider 720 moves in the push-in direction and the pressure of the fluid accommodated in the fluid accommodation portion 765 of the fluid container 760 becomes a predetermined target pressure value.

Thereafter, when the operator inputs an ON signal of the fluid supply ready switch 782 to the pump control unit 710 by pushing the fluid supply ready switch 782, and the pressure of the fluid in the fluid accommodation portion 765 enters a specific range (hereinafter, also referred to as a rough window) for the target pressure value, the pump control unit 710 is brought into a fluid suppliable state in which the fluid is allowed to be supplied from the pump 700 to the pulsation generator 100.

When the pump control unit 710 is in a fluid suppliable state, and the operator inputs an ON signal of the priming switch 783 to the pump control unit 710 by operating the priming switch 783, the pump control unit 710 starts a priming process. The priming process is a process by which a fluid channel from the fluid container 760 to the connection tube 25 and to a fluid ejection opening 212 of the pulsation generator 100 is filled up with the fluid.

When the priming process starts, the pump control unit 710 opens the pinch valve 750, and starts moving the slider 720 in the push-in direction at the same time or substantially the same time (for example, a time gap of approximately several milliseconds) as when the pinch valve 750 is opened. The slider 720 moves at a predetermined speed in such a manner that a constant amount of the fluid per unit time is supplied from the fluid container 760. The priming process is performed until a predetermined amount of time required to complete the priming process has elapsed (or the slider 720 moves by a predetermined distance), or the operator inputs an OFF signal of the priming switch 783 by operating the priming switch 783.

Accordingly, a predetermined amount of the fluid in the fluid accommodation portion 765 is supplied at a predetermined flow speed (the amount of discharge of the fluid per unit time) from the pump 700, the connection tube 25 from the pinch valve 750 to the pulsation generator 100 is filled up with the fluid, and the fluid chamber 501 of the pulsation generator 100, the fluid ejection tube 200 and the like are filled up with the fluid. Air present in the connection tube 25 or the pulsation generator 100 prior to the start of the priming process is released to the atmosphere via the nozzle 211 of the pulsation generator 100 as the fluid flows into the connection tube 25 or the pulsation generator 100.

The pump control unit 710 pre-stores the predetermined speed, the predetermined distance, and the predetermined amount of time in relation to the movement of the slider 720 during the priming process.

As such, the priming process is completed.

Subsequently, when the operator inputs an ON signal of the flushing switch 628 to the drive control unit 600 by operating the flushing switch 628, the drive control unit 600 and the pump control unit 710 start a deaeration process.

The deaeration process is a process by which air bubbles remaining in the connection tube 25 or the pulsation generator 100 are discharged via the nozzle 211 of the pulsation generator 100.

In the deaeration process, in a state in which the pinch valve 750 is opened, the pump control unit 710 moves the slider 720 in the push-in direction at the predetermined speed in such a manner that a constant amount of the fluid per unit time is supplied from the fluid container 760, and the fluid is supplied to the pulsation generator 100. The drive control unit 600 drives the piezoelectric element 401 of the pulsation generator 100 in conjunction with the discharge of the fluid by the pump 700, and thereby the pulsation generator 100 ejects the fluid. Accordingly, air bubbles remaining in the connection tube 25 or the pulsation generator 100 are discharged via the nozzle 211 of the pulsation generator 100. The deaeration process is performed until a predetermined amount of time has elapsed (or the slider 720 moves by a predetermined distance), or the operator inputs an OFF signal of the flushing switch 628 by operating the flushing switch 628.

The drive control unit 600 and the pump control unit 710 pre-store the predetermined speed, the predetermined distance, and the predetermined amount of time in relation to the movement of the slider 720 during the deaeration process.

When the deaeration process is completed, the pump control unit 710 closes the pinch valve 750, and detects the pressure of the fluid accommodated in the fluid accommodation portion 765 of the fluid container 760. The pump control unit 710 performs a control operation in which the position of the slider 720 is adjusted in order for the pressure to become the target pressure value.

Thereafter, when the pressure of the fluid in the fluid accommodation portion 765 enters a specific range (a rough window) for the target pressure value, the pump control unit 710 is brought into a fluid ejectable state in which the fluid can be ejected in the form of a pulsed flow from the pulsation generator 100.

In this state, when the operator inputs an ON signal of the pulsation generator start-up switch 625 to the drive control unit 600 by operating the pulsation generator start-up switch 625 via the feet, the pump control unit 710 opens the pinch valve 750 in response to signals transmitted from the drive control unit 600, and starts the supply of the fluid to the pulsation generator 100 by moving the slider 720 at a predetermined speed in the push-in direction at the same time or substantially the same time (for example, a time gap of approximately several milliseconds) as when the pinch valve 750 is opened. In contrast, the drive control unit 600 generates a pulsed flow by starting the driving of the piezoelectric element 401 and changing the volume of the fluid chamber 501. Accordingly, a pulsed flow of the fluid is ejected at a high speed via the nozzle 211 at the tip of the pulsation generator 100.

Thereafter, when the operator inputs an OFF signal of the pulsation generator start-up switch 625 to the drive control unit 600 by operating the pulsation generator start-up switch 625 via the feet, the drive control unit 600 stops the driving of the piezoelectric element 401. The pump control unit 710 stops the movement of the slider 720 in response to signals transmitted from the drive control unit 600, and closes the pinch valve 750. As such, the pulsation generator 100 stops the ejection of the fluid.

In the embodiment, although the fluid container 760 is configured as a syringe barrel including the syringe 761 and the plunger 762, the fluid container 760 may have other forms. For example, the fluid container 760 may be an infusion bag containing a fluid. In this case, the infusion bag may be mounted as fluid container 760 in the fluid container mounting unit 770. After an opening provided in the infusion bag in order to take out the fluid inside the infusion bag is combined with the connection tube 25, the infusion bag is pressed by a mechanism for pressing the infusion bag from the surrounding, such that the fluid is supplied from the infusion bag inside the pump 700 to the pulsation generator 100.

In addition, in the embodiment, the drive control unit 600 is provided separately from the pump 700 and the pulsation generator 100; however, the drive control unit 600 may be provided integrally with the pump 700.

When the practitioner performs an operation using the fluid ejection device 1, the practitioner grasps the pulsation generator 100. Accordingly, the connection tube 25 up to the pulsation generator 100 is preferably as flexible as possible. For this reason, a flexible thin tube is used as the connection tube 25, and a fluid discharge pressure of the pump 700 is preferably set to a low pressure in a pressure range in which the fluid can be supplied to the pulsation generator 100. For this reason, the discharge pressure of the pump 700 is set to approximately 0.3 atm (0.03 MPa) or less.

In particular, in a case where a malfunction of an apparatus may lead to a serious accident, for example, for a brain surgery, it is necessary to prevent the cutting of the connection tube 25 from causing the ejection of the fluid at a high pressure, and also, for this reason, the discharge pressure of the pump 700 is required to be set to a low pressure.

### Pulsation Generator

Subsequently, the structure of the pulsation generator 100 according to the embodiment will be described.

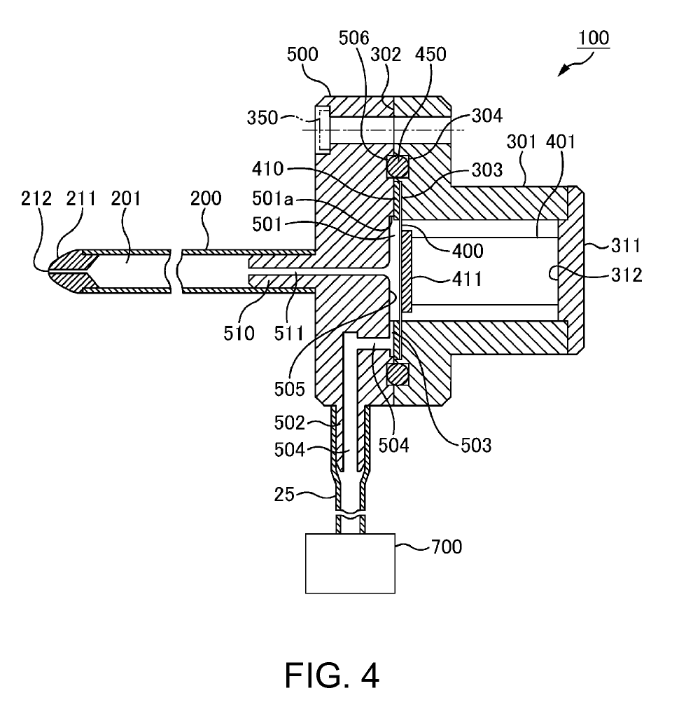

FIG. 4 is a cross-sectional view illustrating the structure of the pulsation generator 100 according to the embodiment. In FIG. 4, the pulsation generator 100 includes a pulse generation unit that generates the pulsation of the fluid, and is connected to the fluid ejection tube 200 having a connection channel 201 as a channel through which the fluid is discharged.

In the pulsation generator 100, an upper case 500 and a lower case 301 are screwed together with four fixation screws 350 (not illustrated) while the respective facing surfaces thereof are bonded to each other. The lower case 301 is a cylindrical member having a flange, and one end portion of the lower case 301 is sealed with a bottom plate 311. The piezoelectric element 401 is provided in an inner space of the lower case 301.

The piezoelectric element 401 is a stack-type piezoelectric element, and acts as an actuator. One end portion of the piezoelectric element 401 is firmly fixed to the diaphragm 400 via an upper plate 411, and the other end portion is firmly fixed to an upper surface 312 of the bottom plate 311.

The diaphragm 400 is made of a circular disc-like thin metal plate, and a circumferential edge portion of the diaphragm 400 is firmly fixed to a bottom surface of a concave portion 303 in the lower case 301 while being in close contact with the bottom surface of the concave portion 303. When drive signals are input to the piezoelectric element 401 that acts as a volume change unit, the piezoelectric element 401 changes the volume of the fluid chamber 501 via the diaphragm 400 through the extension and contraction thereof.

A reinforcement plate 410 is provided in such a manner as to be stacked on an upper surface of the diaphragm 400, and is made of a circular disc-like thin metal plate having an opening at the center thereof.

The upper case 500 has a concave portion formed in a center portion of the surface facing the lower case 301, and the fluid chamber 501 is a rotator-shaped space formed by this concave portion and the diaphragm 400 and filled with the fluid. That is, the fluid chamber 501 is a space enveloped by a sealing surface 505 and an inner circumferential side wall 501a of the concave portion of the upper case 500, and the diaphragm 400. An outlet channel 511 is drilled in an approximately center portion of the fluid chamber 501.

The outlet channel 511 passes through the outlet channel tube 510 from the fluid chamber 501 to an end portion of an outlet channel tube 510 provided in such a manner as to protrude from one end surface of the upper case 500. A connection portion between the outlet channel 511 and the sealing surface 505 of the fluid chamber 501 is smoothly rounded so as to reduce fluid resistance.

In the embodiment (refer to FIG. 4), the fluid chamber 501 has a substantially cylindrical shape having sealed opposite ends; however, the fluid chamber 501 may have a conical shape, a trapezoidal shape, a hemispherical shape, or the like in a side view, and the shape of the fluid chamber 501 is not limited to a cylindrical shape. For example, when the connection portion between the outlet channel 511 and the sealing surface 505 has a funnel shape, air bubbles in the fluid chamber 501 (to be described later) are easily discharged.

The fluid ejection tube 200 is connected to the outlet channel tube 510. The connection channel 201 is drilled in the fluid ejection tube 200, and the diameter of the connection channel 201 is larger than that of the outlet channel 511. In addition, the tube thickness of the fluid ejection tube 200 is formed so as to have a range of rigidity in which the fluid ejection tube 200 does not absorb pressure pulsation of the fluid.

The nozzle 211 is inserted into the tip end portion of the fluid ejection tube 200. A fluid ejection opening 212 is drilled in the nozzle 211. The diameter of the fluid ejection opening 212 is smaller than that of the connection channel 201.

An inlet channel tube (a fluid intake port) 502 is provided in such a manner as to protrude from a side surface of the upper case 500, and is inserted into the connection tube 25 through which the fluid is supplied from the pump 700. A connection channel 504 for the inlet channel is drilled in the inlet channel tube 502. The connection channel 504 communicates with an inlet channel 503. The inlet channel 503 is formed in a groove shape in a circumferential edge portion of the sealing surface 505 of the fluid chamber 501, and communicates with the fluid chamber 501.

A packing box 304 and a packing box 506 are respectively formed in the bonded surfaces of the lower case 301 and the upper case 500 at positions separated from an outer circumferential direction of the diaphragm 400, and a ring-shaped packing 450 is mounted in a space formed by the packing boxes 304 and 506.

Here, when the upper case 500 and the lower case 301 are assembled together, the circumferential edge portion of the diaphragm 400 is in close contact with a circumferential edge portion of the reinforcement plate 410 due to the circumferential edge portion of the sealing surface 505 of the upper case 500 and the bottom surface of the concave portion 303 of the lower case 301. At this time, the packing 450 is pressed by the upper case 500 and the lowercase 301, and thereby the fluid is prevented from leaking from the fluid chamber 501.

Since the inner pressure of the fluid chamber 501 becomes a high pressure of 30 atm (3 MPa) or greater during the discharge of the fluid, the fluid may slightly leak from the respective connections between the diaphragm 400, the reinforcement plate 410, the upper case 500, and the lower case 301; however, the leakage of the fluid is prevented due to the packing 450.

As illustrated in FIG. 4, in the case where the packing 450 is provided, since the packing 450 is compressed due to the pressure of the fluid leaking from the fluid chamber 501 at a high pressure, and is strongly pressed against the respective walls of the packing boxes 304 and 506, it is possible to more reliably prevent the leakage of the fluid. For this reason, it is possible to maintain a considerable increase in the inner pressure of the fluid chamber 501 during the driving of the pulsation generator 100.

Subsequently, the inlet channel 503 formed in the upper case 500 will be described with reference to the drawings in more detail.

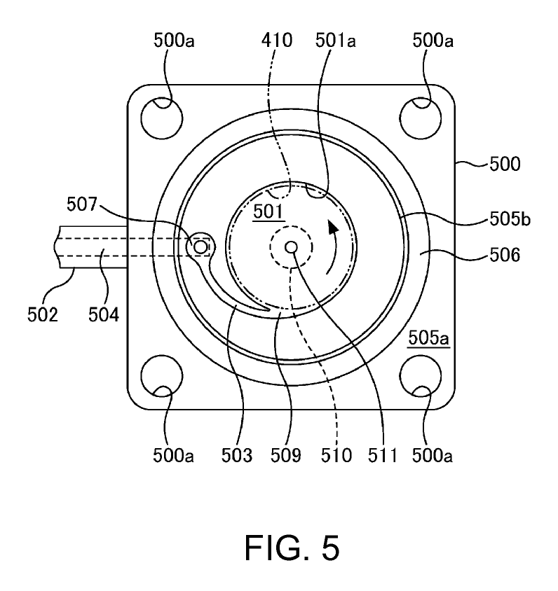

FIG. 5 is a plan view illustrating the shape of the inlet channel 503, and FIG. 5 illustrates the shape of the upper case 500 when the surface of the upper case 500 bonded to the lower case 301 is seen.

In FIG. 5, the inlet channel 503 is formed in a groove shape in the circumferential edge portion of the sealing surface 505 of the upper case 500.

One end portion of the inlet channel 503 communicates with the fluid chamber 501, and the other end portion communicates with the connection channel 504. A fluid sump 507 is formed in a connection portion between the inlet channel 503 and the connection channel 504. A connection portion between the fluid sump 507 and the inlet channel 503 is smoothly rounded, and thereby fluid resistance is reduced.

The inlet channel 503 communicates with the fluid chamber 501 in a substantially tangential direction with respect to an inner circumferential side wall 501a of the fluid chamber 501. The fluid supplied from the pump 700 (refer to FIG. 1) at a predetermined pressure flows along the inner circumferential side wall 501a (in a direction illustrated by the arrow in FIG. 5), and generates a swirl flow in the fluid chamber 501. The swirl flow is pushed against the inner circumferential side wall 501a due to a centrifugal force associated with the swirling of the fluid, and air bubbles in the fluid chamber 501 are concentrated in a center portion of the swirl flow.

The air bubbles concentrated in the center portion are discharged via the outlet channel 511. For this reason, the outlet channel 511 is preferably provided in the vicinity of the center of the swirl flow, that is, in an axial center portion of a rotor shape.

As illustrated in FIG. 5, the inlet channel 503 is curved. The inlet channel 503 may communicate with the fluid chamber 501 while not being curved but being linearly formed; however, when the inlet channel 503 is curved, a channel length is increased, and a desired inertance (to be described later) is obtained in a small space.

As illustrated in FIG. 5, the reinforcement plate 410 is provided between the diaphragm 400 and the circumferential edge portion of the sealing surface 505, in which the inlet channel 503 is formed. The reinforcement plate 410 is provided so as to improve the durability of the diaphragm 400. Since a cut-out connection opening 509 is formed in a connection portion between the inlet channel 503 and the fluid chamber 501, when the diaphragm 400 is driven at a high frequency, stress may be concentrated in the vicinity of the connection opening 509, and thereby a fatigue failure may occur in the vicinity of the connection opening 509. It is possible to prevent stress from being concentrated on the diaphragm. 400 by providing the reinforcement plate 410 with an opening not having a cut-out portion and being continuously formed.

Four screw holes 500a are respectively provided in outer circumferential corner portions of the upper case 500, and the upper case 500 and the lower case 301 are bonded to each other via screwing at the positions of the screw holes.

It is possible to firmly fix the reinforcement plate 410 and the diaphragm 400 in an integrally stacked state by bonding together the reinforcement plate 410 and the diaphragm 400, which is not illustrated. An adhesive method using an adhesive, a solid-state diffusion bonding method, a welding method, or the like may be used so as to firmly fix together the reinforcement plate 410 and the diaphragm 400; however, the respective bonded surfaces of the reinforcement plate 410 and the diaphragm 400 are preferably in close contact with each other.

### Operation of Pulsation Generator

Subsequently, an operation of the pulsation generator 100 according to the embodiment will be described with reference to FIGS. 1 to 5. The pulsation generator 100 according to the embodiment discharges the fluid due to a difference between an inertance L1 (may be referred to as a combined inertance L1) of the inlet channel 503 side and an inertance L2 (may be referred to as a combined inertance L2) of the outlet channel 511 side.

### Inertance

First, the inertance will be described.

An inertance L is expressed by L=ρ×h/S, and here, ρ is the density of a fluid, S is the cross-sectional area of a channel, and h is a channel length. When ΔP is a differential pressure of the channel, and Q is a flow rate of the fluid flowing through the channel, it is possible to deduce a relationship ΔP=L×dQ/dt by modifying an equation of motion in the channel using the inertance L.

That is, the inertance L indicates a degree of influence on a change in flow rate with time, and a change in flow rate with time decreases to the extent that the inertance L is large, and a change in flow rate with time increases to the extent that the inertance L is small.

Similar to a parallel connection or a series connection of inductances in an electric circuit, it is possible to calculate a combined inertance with respect to a parallel connection of a plurality of channels or a series connection of a plurality of channels having different shapes by combining an inertance of each of the channels.

Since the diameter of the connection channel 504 is set to be larger much than that of the inlet channel 503, the inertance L1 of the inlet channel 503 side can be calculated from a boundary of the inlet channel 503. At this time, since the connection tube 25 that connects the pump 700 and the inlet channel 503 is flexible, the connection tube 25 may not be taken into consideration in calculating the inertance L1.

Since the diameter of the connection channel 201 is larger much than that of the outlet channel 511, and the tube (tube wall) thickness of the fluid ejection tube 200 is thin, the connection tube 25 and the fluid ejection device 1 have a negligible influence on the inertance L2 of the outlet channel 511 side. Accordingly, the inertance L2 of the outlet channel 511 side may be replaced with an inertance of the outlet channel 511.

The rigidity of the tube wall thickness of the fluid ejection tube 200 is sufficient to propagate the pressure of the fluid.

In the embodiment, a channel length and a cross-sectional area of the inlet channel 503 and a channel length and a cross-sectional area of the outlet channel 511 are set in such a manner that the inertance L1 of the inlet channel 503 side is greater than the inertance L2 of the outlet channel 511 side.

### Ejection of Fluid

Subsequently, an operation of the pulsation generator 100 will be described.

The pump 700 supplies the fluid to the inlet channel 503 at a predetermined pressure. As a result, when the piezoelectric element 401 is not operated, the fluid flows into the fluid chamber 501 due to a difference between a discharge force of the pump 700 and a fluid resistance value for the entirety of the inlet channel 503 side.

Here, in a case where the inertance L1 of the inlet channel 503 side and the inertance L2 of the outlet channel 511 side are considerably large, when a drive signal is input to the piezoelectric element 401, and the piezoelectric element 401 extends rapidly, the inner pressure of the fluid chamber 501 increases rapidly, and reaches several tens of atmosphere.

Since the inner pressure of the fluid chamber 501 is larger much than the pressure applied to the inlet channel 503 by the pump 700, the flow of the fluid from the inlet channel 503 to the fluid chamber 501 decreases due to the pressure, and the flow of the fluid out of the outlet channel 511 increases.

Since the inertance L1 of the inlet channel 503 is larger than the inertance L2 of the outlet channel 511, an increase in a flow rate of the fluid discharged from the outlet channel 511 is larger than a decrease in a flow rate of the fluid flowing from the inlet channel 503 into the fluid chamber 501. Accordingly, the fluid is discharged in the form of a pulsed flow to the connection channel 201, that is, a pulsed flow occurs. Discharge pressure pulsation propagates in the fluid ejection tube 200, and the fluid is ejected via the fluid ejection opening 212 of the nozzle 211 at the tip end.

Here, since the diameter of the fluid ejection opening 212 of the nozzle 211 is smaller than that of the outlet channel 511, a pulsed flow of the fluid is ejected as droplets at a higher pressure and speed.

In contrast, immediately after a pressure increase, the inner pressure of the fluid chamber 501 becomes negative due to interaction between a decrease in the amount of inflow of the fluid from the inlet channel 503 and an increase in the amount of outflow of the fluid from the outlet channel 511. As a result, after a predetermined amount of time has elapsed, due to both of the pressure of the pump 700 and the negative inner pressure of the fluid chamber 501, the fluid flows from the inlet channel 503 into the fluid chamber 501 again at the same speed as that before the operation of the piezoelectric element 401.

When the piezoelectric element 401 extends after the flow of the fluid from the inlet channel 503 is restored, it is possible to continuously eject the fluid in the form of a pulsed flow via the nozzle 211.

### Discharge of Air Bubbles

Subsequently, an operation of discharging air bubbles from the fluid chamber 501 will be described.

As described above, the inlet channel 503 communicates with the fluid chamber 501 via a path that approaches the fluid chamber 501 while swirling around the fluid chamber 501. The outlet channel 511 is provided in the vicinity of a rotational axis of a substantially rotor-shaped fluid chamber 501.

For this reason, the fluid flowing from the inlet channel 503 into the fluid chamber 501 swirls along the inner circumferential side wall 501a of the fluid chamber 501. The fluid is pushed against the inner circumferential side wall 501a of the fluid chamber 501 due to a centrifugal force, and air bubbles contained in the fluid are concentrated in the center portion of the fluid chamber 501, and are discharged via the outlet channel 511.

Accordingly, even when a small amount of the volume of the fluid chamber 501 is changed in association with the operation of the piezoelectric element 401, it is possible to obtain a sufficient pressure increase while a pressure pulsation is not adversely affected.

In the embodiment, since the pump 700 supplies the fluid to the inlet channel 503 at a predetermined pressure, even when the driving of the pulsation generator 100 is stopped, the fluid is supplied to the inlet channel 503 and the fluid chamber 501. Accordingly, it is possible to start an initial operation without an aid of a prime operation.

Since the fluid is ejected via the fluid ejection opening 212 having a diameter smaller than that of the outlet channel 511, an inner fluid pressure is increased higher than that of the outlet channel 511, and thereby it is possible to eject the fluid at a high speed.

Since the rigidity of the fluid ejection tube 200 is sufficient to transmit a pulsation of the fluid from the fluid chamber 501 to the fluid ejection opening 212, it is possible to eject the fluid in the form of a desired pulsed flow without disturbing pressure propagation of the fluid from the pulsation generator 100.

Since the inertance of the inlet channel 503 is set to be larger than that of the outlet channel 511, an increase in the amount of outflow of the fluid from the outlet channel 511 is larger than a decrease in the amount of inflow of the fluid from the inlet channel 503 into the fluid chamber 501, and it is possible to discharge the fluid into the fluid ejection tube 200 in the form of a pulsed flow. Accordingly, a check valve is not required to be provided in the inlet channel 503, it is possible to simplify the structure of the pulsation generator 100, it is easy to clean the inside of the pulsation generator 100, and it is possible to remove a potential durability problem associated with the use of the check valve.

Since the respective inertances of both of the inlet channel 503 and the outlet channel 511 are set to be considerably large, it is possible to rapidly increase the inner pressure of the fluid chamber 501 by rapidly reducing the volume of the fluid chamber 501.

Since the piezoelectric element 401 as a volume change unit and the diaphragm 400 are configured so as to generate a pulsation, it is possible to simplify the structure of the pulsation generator 100 and to reduce the size of the pulsation generator 100 in association therewith. It is possible to set the maximum frequency of a change in the volume of the fluid chamber 501 to a high frequency of 1 KHz or greater, and the pulsation generator 100 is optimized to eject a pulsed flow of the fluid at a high speed.

In the pulsation generator 100, since the inlet channel 503 generates a swirl flow of the fluid in the fluid chamber 501, the fluid in the fluid chamber 501 is pushed in an outer circumferential direction of the fluid chamber 501 due to a centrifugal force, air bubbles contained in the fluid are concentrated in the center portion of the swirl flow, that is, in the vicinity of the axis of the substantially rotor shape, and thereby it is possible to discharge the air bubbles via the outlet channel 511 provided in the vicinity of the axis of the substantially rotor shape. For this reason, it is possible to prevent a decrease in pressure amplitude associated with the stagnation of air bubbles in the fluid chamber 501, and it is possible to continuously and stably drive the pulsation generator 100.

Since the inlet channel 503 is formed in such a manner as to communicate with the fluid chamber 501 via the path that approaches the fluid chamber 501 while swirling around the fluid chamber 501, it is possible to generate a swirl flow without adopting a structure dedicated for swirling the fluid in the fluid chamber 501.

Since the groove-shaped inlet channel 503 is formed in the outer circumferential edge portion of the sealing surface 505 of the fluid chamber 501, it is possible to form the inlet channel 503 (a swirl flow generation unit) without increasing the number of components.

Since the reinforcement plate 410 is provided on the upper surface of the diaphragm 400, the diaphragm 400 is driven with respect to an outer circumference (a fulcrum) of the opening of the reinforcement plate 410, and thereby the concentration of stress is unlikely to occur, and it is possible to improve the durability of the diaphragm 400.

When corners of the surface of the reinforcement plate 410 bonded to the diaphragm 400 are rounded, it is possible to further reduce the concentration of stress on the diaphragm 400.

When the reinforcement plate 410 and the diaphragm 400 are firmly and integrally fixed together while being stacked on each other, it is possible to improve the assemblability of the pulsation generator 100, and it is possible to reinforce the outer circumferential edge portion of the diaphragm 400.

Since the fluid sump 507 for the stagnation of the fluid is provided in the connection portion between the connection channel 504 on an inlet side for supplying the fluid from the pump 700 and the inlet channel 503, it is possible to prevent the inertance of the connection channel 504 from affecting the inlet channel 503.

In the respective bonded surfaces of the lower case 301 and the upper case 500, the ring-shaped packing 450 is provided at the position separated from the outer circumferential direction of the diaphragm 400, and thereby it is possible to prevent the leakage of the fluid from the fluid chamber 501, and to prevent a decrease in the inner pressure of the fluid chamber 501.

### Failure Detection of Pressure Sensor

As described above, as for the fluid ejection device 1 of the present embodiment, when the pressure of the fluid contained in the fluid accommodation portion 765 is in a range (rough window) defined with respect to a predetermined target pressure value, in a liquid feed-possible state of allowing the feed of the fluid from the pump 700 to the pulsation generator 100, if an operator inputs the ON signal of the pulsation generator start-up switch 625 to the drive control unit 600, the drive control unit 600 starts the drive of the piezoelectric element 401, and a pulsed flow of the fluid is ejected at a high speed out of the nozzle 211 at the tip of the pulsation generator 100.

As described above, since the fluid ejection device 1 controls in such a manner that the pressure of the fluid in the fluid accommodation portion 765 is in the rough window, the fluid is ejected from the pulsation generator 100 at a proper intensity.

Therefore, when a failure occurs in the pressure sensor 722 in which the output characteristics of the pressure sensor 722 detecting the pressure of the fluid in the fluid accommodation portion 765 are not regular characteristics, the pressure of the fluid in the fluid accommodation portion 765 is controlled so as to be in a range different from the rough window, and there is a possibility that the fluid is ejected from the pulsation generator 100 at a unexpected strength of the operator.

In the embodiment, the output characteristics of the pressure sensor 722 refers to the correspondence between the pressure applied to the pressure sensor 722 and the level of the detection signal output from the pressure sensor 722.

In the embodiment, the normal state of the pressure sensor 722 refers to a state where the output characteristics of the pressure sensor 722 are within a predetermined range that is normal. The output characteristics in which the correspondence between the pressure applied to the pressure sensor 722 and the level of the detection signal output from the pressure sensor 722 is within the predetermined range are referred to as regular characteristics.

The failure of the pressure sensor 722 refers to a case where the pressure sensor 722 is not normal, that is, a case where the output characteristics of the pressure sensor 722 are not regular characteristics.

The fluid ejection device 1 according to the present embodiment is capable of detecting the failure in which the output characteristics of the pressure sensor 722 are no longer the regular characteristics.

Although details will be described later, in the fluid ejection device 1 according to the present embodiment, for example, pressing to the fluid accommodation portion 765 is performed by moving the slider (moving object) 720 at a predetermined speed in the pressing direction in a state of closing the pinch valve (channel opening and closing unit) 750, and the failure of the pressure sensor 722 is determined depending on whether or not a time required until the pressure is the second determination value after the pressure detected by the pressure sensor 722 is equal to or greater than the first determination value is equal to or greater than a predetermined determination time.

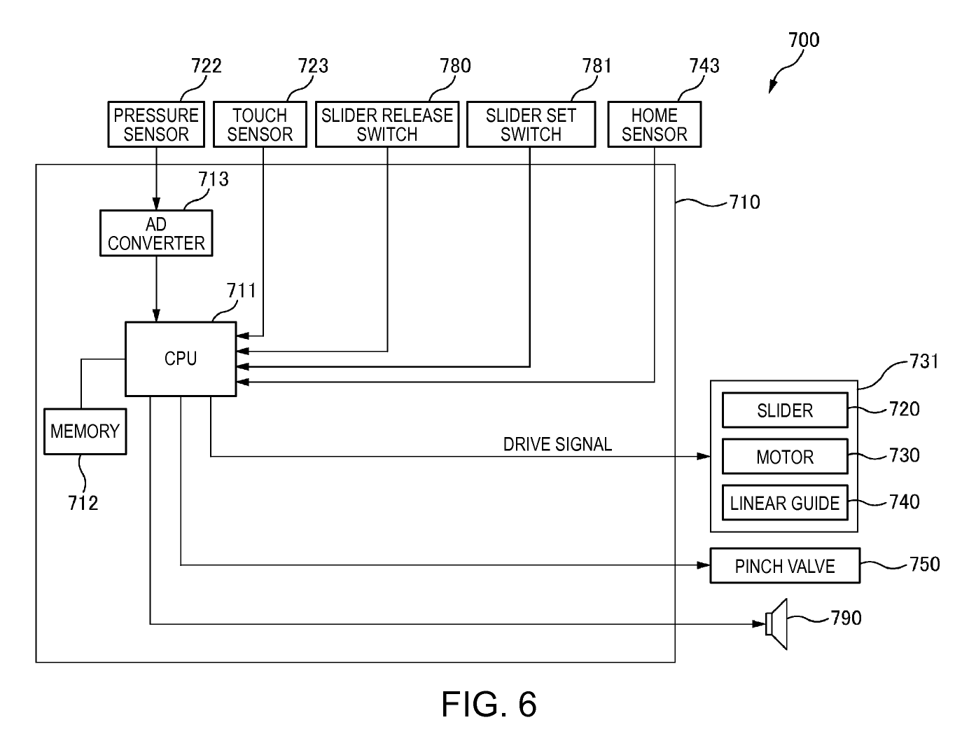

A failure detection process by the pressure sensor 722 in the fluid ejection device 1 according to the embodiment will be specifically described with reference to FIG. 6 to FIG. 8.

First, a configuration of the pump control unit (control unit) 710 will be described with reference to FIG. 6.

The pump control unit 710 is configured to include a central processing unit (CPU) 711, a memory 712, and an analog/digital (A/D) converter 713.

The pump control unit 710 receives a detection signal of a level corresponding to the pressure when the fluid pressing unit 731 presses the fluid accommodation portion 765 of the fluid container 760 from the pressure sensor 722, and controls the fluid pressing unit 731. For example, when an ON signal of the slider set switch 781 is input, the pump control unit 710 moves the slider 720 in a pressing direction by outputting a predetermined drive signal to the fluid pressing unit 731 and driving the motor 730, in a state where the pinch valve 750 is closed, and controls the motor 730 such that the inner pressure of the fluid accommodation portion 765 that is detected by the pressure sensor 722 is a predetermined target pressure value. The fluid pressing unit 731 is configured to have the slider 720, the motor 730, and the linear guide 740.

The CPU 711 controls the entirety of the pump control unit 710, and realizes various functions of the embodiment by executing a program made of codes for executing various operations stored in the memory 712.

The memory 712 stores various pieces of data in addition to the program. For example, the memory 712 stores first determination value level data (for example, a voltage value) indicating a level (for example, a voltage) corresponding to the above-mentioned first determination value (a pressure value), second determination value level data indicating a level corresponding to the second determination value, or a first determination time and a second determination time which are described later, or the like, in addition to the program.

The AD converter 713 receives a detection signal output from the pressure sensor 722, and outputs data indicative of a level of the detection signal. Specifically, the pressure sensor 722 detects the pressure when the slider 720 presses the fluid accommodation portion 765, and outputs a detection signal of a level (for example, a voltage) corresponding to the pressure, but the AD converter 713 outputs detected level data (for example, a voltage value) indicative of the level of the detection signal output from the pressure sensor 722.

### First Failure Detection Process

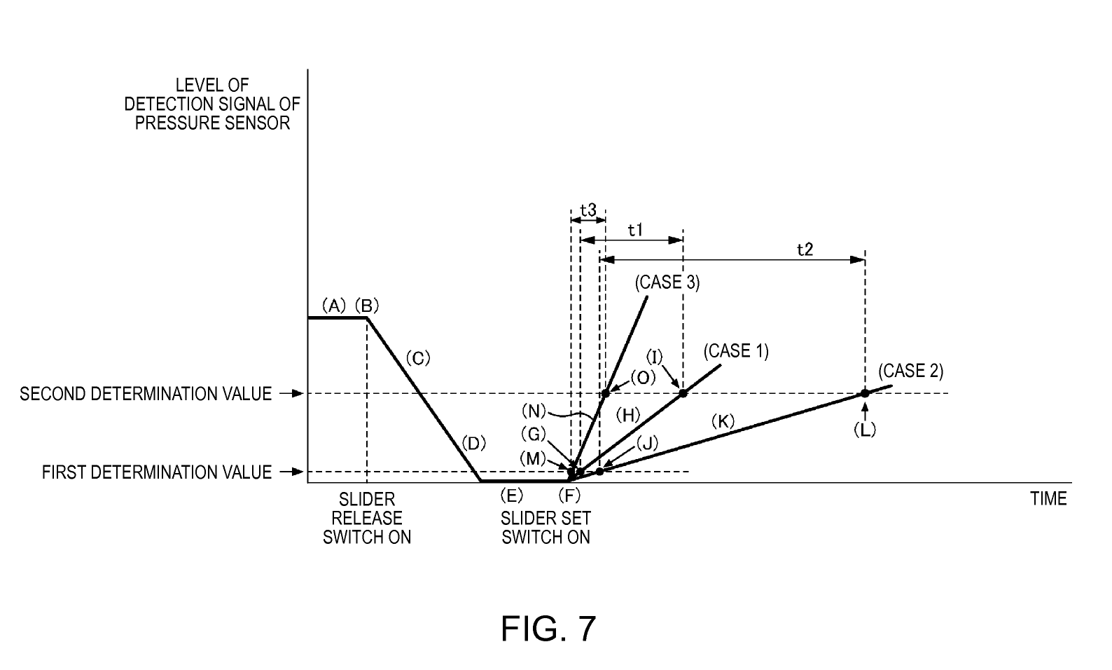

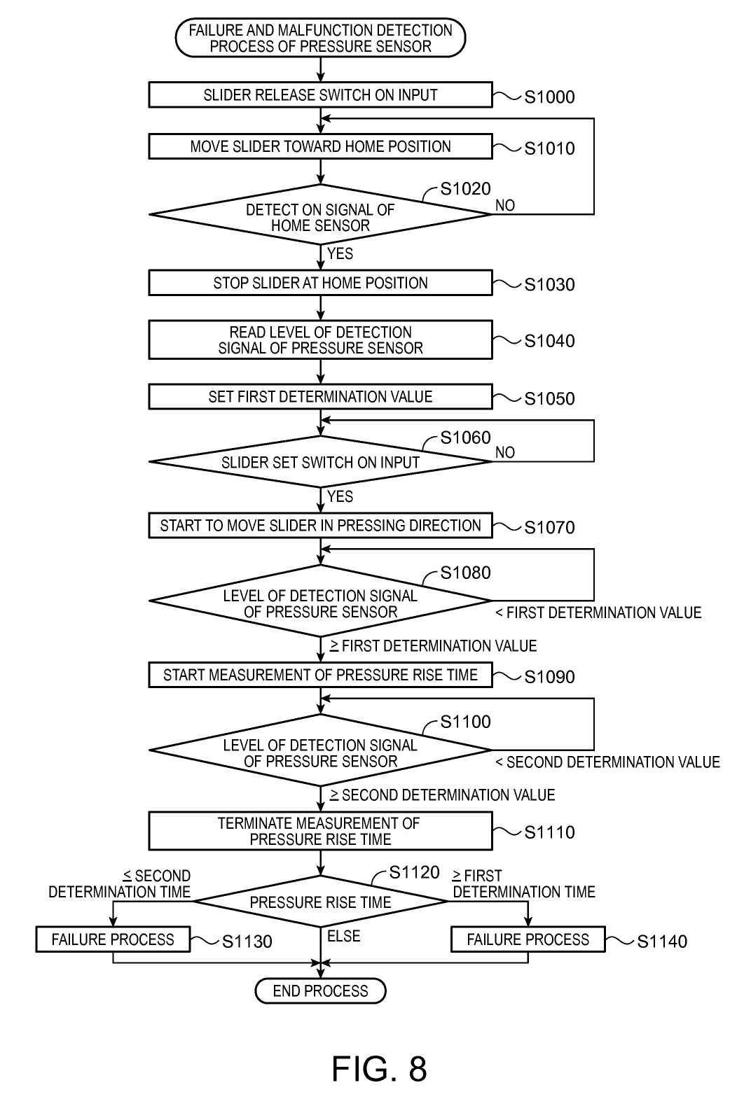

Hereinafter, a flow of a first failure detection process of a pressure sensor 722 according to the embodiment will be described with reference to FIG. 7 and FIG. 8. In addition, FIG. 7 is a diagram illustrating a change of an inner pressure of the fluid accommodation portion 765, and FIG. 8 is a flowchart illustrating a flow of the first failure detection process.

In the following description, the flow of the first failure detection process will be described from when the ON signal of the slider release switch 780 is input to the signal pump control unit 710.

First, the CPU 711 detects the input of the ON signal of the slider release switch 780 ((B) in FIG. 7, S1000 in FIG. 8). Then, the CPU 711 moves the slider 720 toward the home position in an opposite direction of the pressing direction, by outputting a predetermined drive signal to the fluid pressing unit 731 (S1010 in FIG. 8). Then, the plunger 762 moves in the opposite direction of the pressing direction due to the pressure of the fluid in the fluid accommodation portion 765, and therefore, the pressure of the fluid in the fluid accommodation portion 765 decreases ((C) in FIG. 7).

While the CPU 711 moves the slider 720 toward the home position, if the slider 720 moves away from the plunger 762, the output signal from the touch sensor 723 is changed to OFF ((D) in FIG. 7).

Thereafter, if the CPU 711 continues to move the slider 720 toward the home position in an opposite direction of the pressing direction, the slider 720 reaches the position of the home sensor 743. At this time, the CPU 711 detects the ON signal that is output from the home sensor 743. The CPU 711 stops the slider 720 at the home position ((E) in FIG. 7, S1030 in FIG. 8).

The CPU 711 acquires detection level data (a voltage value) indicating the level (voltage) of the detection signal that is output from the pressure sensor 722 at this time, from the AD converter 713 (S1040 in FIG. 8), and sets first determination value level data (a voltage value) indicating a first determination value (a pressure value) (S1050 in FIG. 8).

The first determination value is used as a reference to start a time measurement (measurement of the pressure rise time) for determining the failure of the pressure sensor 722 when the CPU 711 gradually increases the pressure in the fluid accommodation portion 765, in the following subsequent process. In other words, at the timing when the pressure in the fluid accommodation portion 765 indicated by the detection signal output from the pressure sensor 722 is equal to or greater than the first determination value, the CPU 711 starts the measurement of the pressure rise time.

In this manner, the first determination value is a reference for time measurement start, and can be defined as various values, but when the pressure sensor 722 is in failure, there is a possibility that the level of the detection signal output from the pressure sensor 722 is out of the normal range when the slider 720 is stopped at the home position, such that it is preferred that the first determination value is set to a relative value based on the level of the detection signal output from the pressure sensor 722 when the slider 720 is in the home position.

Thus, even if the output characteristics of the pressure sensor 722 which is the reference when performing the failure detection process of the pressure sensor 722 deviates from the normal characteristics, it is possible to detect the failure of the pressure sensor 722.

Therefore, in this embodiment, the value obtained by adding a predetermined value to the pressure indicated by the detection signal output by the pressure sensor 722 when the slider 720 is in the home position (in other words, the slider 720 is in a predetermined position where the slider 720 does not come into contact with the fluid accommodation portion 765) is assumed as a first determination value.

In order to improve the accuracy of failure detection in the pressure sensor 722, it is preferable to set the widths of the first determination value for starting the measurement of the pressure rise time and the second determination value to be as wide as possible.

Therefore, in this embodiment, the predetermined value is defied such that from when an acceleration period immediately after the slider 720 starts to move in the pressing direction ends and the slider 720 starts to press the plunger 762 at a constant speed, the measurement of the pressure rise time can be started at the earliest possible time after the pressure in the fluid accommodation portion 765 starts to rise. Therefore, in this embodiment, the predetermined value is a value obtained by adding a possible smallest positive value as an increased amount to the maximum error that can be included in the detection signal output from the pressure sensor 722.

By doing so, while eliminating the influence of the error of the pressure sensor 722, it is possible to improve the accuracy of the failure detection of the pressure sensor 722, by starting the measurement of the pressure rise time immediately after the pressure in the fluid accommodation portion 765 has started to rise.

Further, for example, even if the level of the detection signal of the pressure sensor 722 is shifted to be higher by the amount of the maximum error, the first determination value is a much larger value by the increased amount, such that it is possible to prevent a situation where the measurement of pressure rise time starts before starting to press the fluid accommodation portion 765.

It is preferable that the predetermined value used to calculate the first determination value is determined in consideration of not only the error of the pressure sensor 722 but also the error of the AD converter 713 (for example, the quantization error and the like).

The CPU 711 stores the first determination value level data indicating the first determination value that is defined as described above in the memory 712 (S1050 in FIG. 8).

Thereafter, the CPU 711 detects the input of the ON signal of the slider set switch 781 (S1060 in FIG. 8). Then, the CPU 711 moves the slider 720 in a pressing direction, by outputting a predetermined drive signal to the fluid pressing unit 731 (S1070 in FIG. 8). Then, if the slider 720 comes into contact with the plunger 762, an ON signal is output from the touch sensor 723, thereafter, the slider 720 starts to press the plunger 762 ((F) in FIG. 7). Then, the pressure of the fluid in the fluid accommodation portion 765 starts to rise.

When the output characteristics of the pressure sensor 722 are normal, the level of the detection signal from the pressure sensor 722 increases as illustrated in a case 1 in FIG. 7 ((H) in FIG. 7). When the output characteristics of the pressure sensor 722 are not normal, the level of the detection signal from the pressure sensor 722 increases as illustrated in a case 2 in FIG. 7 ((K) in FIG. 7) or a case 3 ((N) in FIG. 7).

In the case 2, the level of the detection signal for the pressure applied to the pressure sensor 722 is shifted to be lower, as compared to the case where the output characteristics are normal. In the case 3, the level of the detection signal for the pressure applied to the pressure sensor 722 is shifted to be higher, as compared to the case where the output characteristics are normal.

If the detection level data indicating the level of the detection signal of the pressure sensor 722 is equal to or greater than the first determination value level data ((G), (J), (M) in FIG. 7, S1080 in FIG. 8), the CPU 711 starts the measurement of a pressure rise time (S1090 in FIG. 8).

Since the CPU 711 continues to move the slider 720, the pressure of the fluid in the fluid accommodation portion 765 continues to rise ((H), (K), (N) in FIG. 7).

If the detection level data indicating the level of the detection signal from the pressure sensor 722 is the second determination value level data ((I), (L), (O) in FIG. 7, S1100 in FIG. 8), the CPU 711 ends the measurement of a pressure rise time (S1110 in FIG. 8).

Similar to the first determination value, the second determination value is set to a relative value based on the level of the detection signal output from the pressure sensor 722 when the slider 720 is in the home position.

When a time (pressure rise time) required until the detection level data is the second determination value level data after the detection level data indicating the level of the detection signal from the pressure sensor 722 is equal to or greater than the first determination value level data is equal to or greater than the first determination time or is equal to or less than the second determination time (S1120 in FIG. 8), the CPU 711 determines that the pressure sensor 722 is in failure, and executes a failure process (S1130 and S1140 in FIG. 8).

In other words, when the pressure rise time (t2 in FIG. 7) is equal to or greater than the first determination time as the case 2 in FIG. 7, the CPU 711 determines that the pressure sensor 722 is in failure (S1120 in FIG. 8).

Even when the pressure rise time (t3 in FIG. 7) is equal to or less than the second determination time as the case 3 in FIG. 7, the CPU 711 determines that the pressure sensor 722 is in failure (S1120 in FIG. 8).

The first determination time is a determination condition for detecting the case where the output characteristics of the pressure sensor 722 are shifted to be lower than in the normal case, as the case 2. The second determination time is a determination condition for detecting the case where the output characteristics of the pressure sensor 722 are shifted to be higher than in the normal case, as the case 3. The first determination time and the second determination time are set in such a manner that the first determination time is longer than the second determination time.

When the pressure rise time (t1 in FIG. 7) is longer than the second determination time, and shorter than the first determination time as the case 1, the CPU 711 determines that the pressure sensor 722 is normal (S1120 in FIG. 8).

When it is determined that the pressure sensor 722 is in failure, the CPU 711 outputs a predetermined alarm in the failure process (S1130 and S1140 in FIG. 8). For example, the CPU 711 outputs a voice message indicating that the pressure sensor 722 is in failure from the speaker 790. Otherwise, the CPU 711 turns on a predetermined warning light (not illustrated).

In such an embodiment, it is possible to quickly inform the operator that the pressure sensor 722 is in failure and further improve the safety of the fluid ejection device 1.

When it is determined that the pressure sensor 722 is in failure, the CPU 711 may release the pinch valve 750 in the failure process (S1130 and S1140 in FIG. 8).

In this manner, even if the pressure in the fluid accommodation portion 765 has become higher than an expected pressure, it is possible to reduce the pressure in the fluid accommodation portion 765 by allowing the fluid in the fluid accommodation portion 765 to flow from the nozzle 211 of the pulsation generator 100, which enables improving the safety of the fluid ejection device 1.

When releasing the pinch valve 750 in the failure process (S1130 and S1140 in FIG. 8), the CPU 711 may cause the fluid ejection device 1 to be in a liquid feed-possible state.

In this manner, even when the pressure sensor 722 is in failure, an operator operates a pulsation generator start-up switch 625 so as to input the ON signal of the pulsation generator start-up switch 625 to the drive control unit 600, which enables a pulsed flow of the fluid to be ejected at a high speed out of the pulsation generator 100.

In this case, for example, an operator checks the strength of the ejection by experimentally ejecting a fluid from the pulsation generator 100, and it is possible for an operator to continue an operation depending on the operator's determination, even when the pressure sensor 722 is in failure.

### Second Failure Detection Process

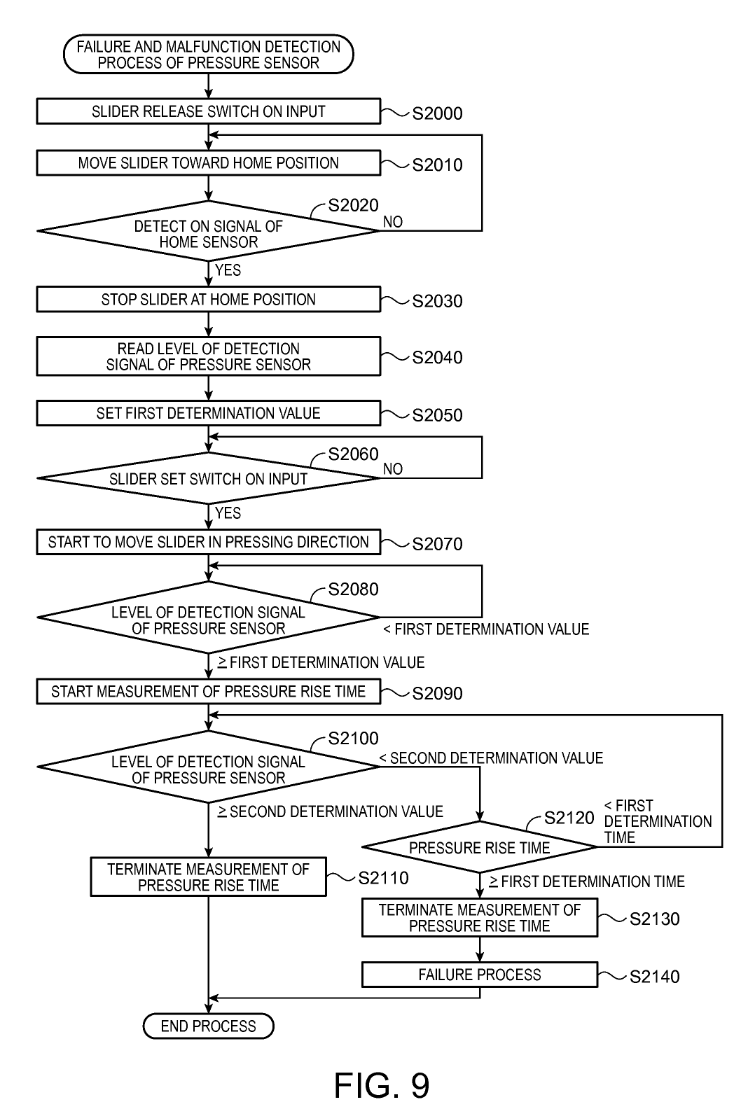

Next, a second failure detection process according to the embodiment will be described with reference to the flowchart illustrated in FIG. 9. The second failure detection process which will be described later can detect a failure of the case 2 illustrated in FIG. 7 in a shorter time.

Since the process of S2000 to S2080 in the flowchart of FIG. 9 is the same as the process of S1000 to S1080 in FIG. 8, the description thereof will be omitted.

Although the CPU 711 starts the measurement of a pressure rise time in S2090, the CPU 711 continues to move the slider 720 as it is, and thus the pressure of the fluid in the fluid accommodation portion 765 continues to rise (similar to (H), (K) in FIG. 7).

At this time, the CPU 711 detects whether or not the detection level data indicating the level of the detection signal from the pressure sensor 722 is the second determination value level data (S2100 in FIG. 9) while moving the slider 720.

Immediately after the measurement of a pressure rise time is started, the detection level data is smaller than the second determination value level data, the CPU 711 proceeds to “<second determination value” from S2100 in FIG. 9. Then, the CPU 711 determines whether or not the pressure rise time is equal to or greater than the first determination time (S2120 in FIG. 9).

When the pressure rise time is less than the first determination time, the CPU 711 determines again whether or not the detection level data is equal to or greater than the second determination value level data (S2100 in FIG. 9).

At this time, when the detection level data is equal to or greater than the second determination value level data, the detection level data is equal to or greater than the second determination value level data before the pressure rise time is the first determination time such that the CPU 711 determines that the pressure sensor 722 is normal, and terminates the measurement of a pressure rise time (S2110 in FIG. 9).

In contrast, at this time, when the detection level data is not equal to or greater than the second determination value level data, in S2120, the CPU 711 determines again whether or not the pressure rise time is equal to or greater than the first determination time (S2120 in FIG. 9).

At this time, if the pressure rise time is equal to or greater than the first determination time, before the detection level data is equal to or greater than the second determination value level data, the pressure rise time is the first determination time, such that it takes the first determination time or more until the detection level data is the second determination value level data after the detection level data is equal to or greater than the first determination value level data. Accordingly, the CPU 711 determines that the pressure sensor 722 is in failure, and terminates the measurement of a pressure rise time and executes the failure process (S2130 and S2140 in FIG. 9).

The contents of the failure process are the same as those of S1130 and S1140 in FIG. 8.

In this manner, in the embodiment, without waiting until the detection level data is equal to or greater than the second determination value level data, it is possible to determine a failure of the pressure sensor 722 at a timing when the pressure rise time becomes the first determination time, and thus it is possible to detect the failure of pressure sensor 722 at a shorter time.

Particularly, when a failure has occurred in which the output characteristics of the pressure sensor 722 are shifted to be lower, the pressure of the fluid ejected from the pulsation generator 100 is shifted to be higher, such that it is possible to detect such a failure at a shorter time, and to further improve the security of the fluid ejection device 1.

### OTHER EMBODIMENTS

The fluid ejection device 1 according to the embodiment, as described below, can also be implemented using the slider 720 having a plurality of pressure sensors 722.

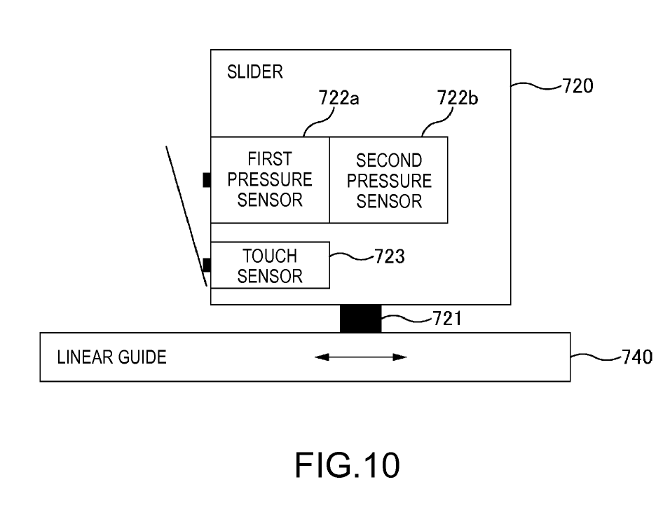

FIG. 10 illustrates a configuration in which the slider 720 includes a first pressure sensor (a first pressure detection unit) 722a and a second pressure sensor (a second pressure detection unit) 722b.

As illustrated in FIG. 10, the slider 720 according to the embodiment is configured in such a manner that the first pressure sensor 722a and the second pressure sensor 722b are arranged so as to be stacked in a pressing direction of the slider 720.

Then, the first pressure sensor 722a and the second pressure sensor 722b are provided at positions where they are clamped by the slider 720 and the plunger 762 when the slider 720 presses the fluid accommodation portion 765.

For this reason, the pressure when the slider 720 presses the fluid accommodation portion 765 is detected by both the first pressure sensor 722a and second pressure sensor 722b, the first detection signal is output from the first pressure sensor 722a, and the second detection signal is output from the second pressure sensor 722b.

When the first detection signal and the second detection signal are input, the AD converter 713 outputs the first detection level data and the second detection level data respectively corresponding to them.

The CPU 711 controls various fluid pressing units 731 described above, by using either or both of the first detection level data and the second detection level data. For example, the CPU 711 moves the slider 720 such that the pressure when the slider 720 presses the fluid accommodation portion 765 is a predetermined target pressure value, by using either or both of the first detection level data and the second detection level data.

In the case of using both the first detection level data and the second detection level data, the CPU 711 may calculate an average value of them and use the average value in the control. In this aspect, for example, even when a noise is mixed to any one of the first detection signal and the second detection signal, and the value of the detection level data of the noise-mixed signal is abruptly changed, the CPU 711 can continue the stable control for the fluid pressing unit 731.

When it is determined that the first pressure sensor 722a is in failure through the first failure detection process or the second failure detection process, which is described above, the CPU 711 continues the control by using the second pressure sensor 722b, and when it is determined that the second pressure sensor 722b is in failure, the CPU 711 continues the control by using the first pressure sensor 722a.

In this aspect, even when the failure occurs in the pressure sensor 722, the fluid ejection device 1 can perform the control so as to continue the ejection of the fluid out of the pulsation generator 100, and it becomes possible to improve the reliability of the fluid ejection device 1.

In this case, the fluid ejection device 1 may output an alarm indicating a failure of the pressure sensor 722, or may not output the alarm. In the former case, the fluid ejection device 1 can continue the control by using a normal one of the pressure sensors 722, but it is possible to quickly inform an operator that the pressure sensor 722 is in failure.

In the latter case, even when a failure occurs in one of the pressure sensors 722, the fluid ejection device 1 can notify that a failure occurs, and thus it is possible to prevent irritating an operator who focuses on an operation. In this case, a fact that a failure occurs in one of the pressure sensors 722 may be stored as log information in the memory 712. Thus, when performing the maintenance of fluid ejection device 1 after the completion of the operation, it is possible to inform the maintenance personnel that the pressure sensor 722 is in failure.

Alternately, for example, when a difference between the first detection level data and the second detection level data is equal to or greater than a predetermined value, the fluid ejection device 1 according to the embodiment may determine that a failure occurs in at least one of the first pressure sensor 722a and the second pressure sensor 722b, and perform the above-described failure process of outputting a predetermined alarm.

In this aspect, even when the first failure detection process and the second failure detection process are not performed, other than the detection of the failure of the pressure sensor 722 through the first failure detection process and the second failure detection process, it is possible to detect the failure of the pressure sensor 722.

Hitherto, the fluid ejection device 1 according to the embodiment has been described in detail, but according to the fluid ejection device 1 according to the embodiment, it is possible to detect the failure of the pressure sensor 722 and realize a safer and high reliable fluid ejection device 1.

The embodiment described above is presented so as to help the understanding of the invention, and does not limit the interpretation of the invention. Modifications and improvements can be made to the invention without departing from the spirit of the invention, and the equivalents are also included in the invention.

What is claimed is:

1. A fluid ejection device comprising:
a fluid container that has a fluid accommodation portion for accommodating a fluid, and a fluid outlet formed in the fluid accommodation portion;
a fluid pressing unit that presses the fluid accommodation portion to cause the fluid to flow from the fluid outlet;
a connection tube, one end of which is connected to the fluid outlet;
a fluid ejection unit that has a fluid intake port connected to the other end of the connection tube, and ejects the fluid taken from the fluid intake port in a pulsed manner;
a channel opening and closing unit that opens and closes a channel of the fluid in the connection tube;
a pressure detection unit that detects a pressure when the fluid pressing unit presses the fluid accommodation portion, and outputs a detection signal of a level corresponding to the pressure; and
a control unit that causes the fluid pressing unit to press the fluid accommodation portion in a state of causing the channel opening and closing unit to close the channel, and determines a failure of the pressure detection unit depending on whether or not a time required until the pressure is a second determination value after the pressure indicated by the detection signal is equal to or greater than a first determination value is equal to or greater than a predetermined determination time.

2. The fluid ejection device according to claim 1,
wherein the fluid pressing unit includes
a moving object that moves in a pressing direction of the fluid accommodation portion, and performs pressing to the fluid accommodation portion, and
a position detection unit that detects a position of the moving object, and
wherein the first determination value is a value that is determined based on the pressure indicated by the detection signal which is output by the pressure detection unit when the moving object is in a predetermined position where the moving object does not come into contact with the fluid accommodation portion.

3. The fluid ejection device according to claim 2,
wherein the first determination value is a sum of the pressure indicated by the detection signal which is output by the pressure detection unit when the moving object is in the predetermined position and a predetermined value which is determined in accordance with an error of the pressure.

4. The fluid ejection device according to claim 2, further comprising:
a first of the pressure detection unit and a second of the pressure detection unit,
wherein the first of the pressure detection unit and the second of the pressure detection unit are arranged so as to be stacked in the pressing direction of the moving object, in a position where the first of the pressure detection unit and the second of the pressure detection unit are clamped by the moving object and the fluid accommodation portion, when the moving object presses the fluid accommodation portion, and
wherein the control unit
controls the movement of the moving object by using either or both of a first of the detection signal which is output from the first of the pressure detection unit and a second of the detection signal which is output from the second of the pressure detection unit in such a manner that a pressure when the moving object presses the fluid accommodation portion is a predetermined target pressure value,
continues the control by using the second of the detection signal when a failure of the first of the pressure detection unit is determined, and
continues the control by using the first of the detection signal when a failure of the second of the pressure detection unit is determined.

5. The fluid ejection device according to claim 1,
wherein the control unit causes the channel opening and closing unit to open the channel, when a failure of the pressure detection unit is determined.

6. The fluid ejection device according to claim 1,
wherein the control unit outputs an alarm indicating a failure of the pressure detection unit, when the failure of the pressure detection unit is determined.
